SEQUENCE LISTING (2 sequences)

SEQ ID NO: 1 - DNA, 33 bp, Artificial
  synthetic DNA enzyme
gccctcccaggctagctacaacgacctcgggct

SEQ ID NO: 2 - DNA, 31 bp, Artificial
  synthetic DNA enzyme
cgcgtccaggctagctacaacgatgcgctcc

(19) United States
(12) Patent Application Publication
(10) Pub. No.: US 2008/0124315 A1
(43) Pub. Date: May 29, 2008

(54) Method of reducing CRF receptor mRNA

(76) Inventors: Steven A. Nanda, Middleton, WI (US); Vaishali P. Bakshi, Madison, WI (US); Patrick H. Roseboom, Madison, WI (US); Ned H. Kalin, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/488,276

(22) Filed: Jul. 18, 2006

Related U.S. Application Data
(63) Continuation of application No. 10/896,544, filed on Jul. 22, 2004, now abandoned.
(60) Provisional application No. 60/489,780, filed on Jul. 24, 2003.

(51) Int. Cl. A61K 38/47; A61P 1/00; A61P 1/04; A61P 9/00; A61K 38/48; A61P 25/24; A61K 38/46

(52) U.S. Cl. 424/94.61; 424/94.6; 424/94.63

(57) ABSTRACT

A method of treating a patient is disclosed. In one embodiment, the method comprises the step of reducing the amount of mRNA encoding corticotropin-releasing factor receptor, wherein a portion of the mRNA encoding corticotropin-releasing factor receptor is destroyed and wherein the amount of corticotrophin releasing factor receptor is reduced.

### CROSS-REFERENCE TO RELATED APPLICATION

This application is a continuation of U.S. application Ser. No. 10/896,544, filed Jul. 22, 2004, which in turn claims priority to U.S. provisional application 60/489,780, filed Jul. 24, 2003, both of which are hereby incorporated by reference herein.

### STATEMENT REGARDING FEDERALLY SPONSORED RESEARCH OR DEVELOPMENT

Not Applicable.

### BACKGROUND OF THE INVENTION

In modern society stress and its consequences are prevalent and result in considerable distress, alterations in physical health and social and occupational functioning. At its extreme, stress can lead to disabling neuropsychiatric problems which include depression, anxiety disorders, post-traumatic stress disorder and other illnesses (Mitchell, Neurosci. Biobehav. Rev. 22:635-651, 1998; Arborelius, et al., J. Endocrinol. 153:185-191, 1999). Recent studies demonstrate the potent effects of stress on the body and brain. For example, chronic and intense stress can result in alterations in the region of the brain that plays an important role in memory (McGaugh and Roozendaal, Curr. Opin. Neurobiol. 12:205-210, 2002). In addition, stress can negatively impact cardiovascular function, immune function and gastrointestinal physiology (Tache, et al., Am. J. Physiol. Gastrointest. Liver Physiol. 280:G173-177, 2001; Beglinger and Degen, Gut Suppl. 1: 145-149, 2002; Coste, et al., Trends Cardiovasc. Med. 12:176-182, 2002; Gasparotto, et al., Physiol. Behav. 76:321-326, 2002; Vanitallie, Metabolism 51:40-45, 2002).

It is estimated that 10% of the population suffers from depression and another 15% from clinically significant anxiety. This high incidence of stress-related problems is reflected by the fact that approximately 50% of visits to primary care doctors are stress and/or psychologically related.

Current treatments for stress and its disorders are highly sought after and include the traditional anti-anxiety drugs like VALIUM and XANAX. More recently newer antidepressants like PROZAC have been used to treat depression, anxiety and other stress related problems. It is estimated that $13 billion was spent last year in the U.S. on drugs like PROZAC. However, these treatments still stiffer from lack of efficacy in approximately 30% of individuals and in those that do respond only roughly 50% of them will return to normal function. In addition, these treatments have bothersome side-effects (50% have marked sexual dysfunction) which make treatment with these drugs unacceptable for many individuals. Since depression and anxiety are recurrent and chronic disorders it is important that patients are comfortable taking their medication over a long period of time. Overactivity of the corticotropin-releasing factor CRF receptor is implicated in depression and anxiety and treatments aimed at this system may be very effective (Reul and Holsboer, Curr. Opin. Pharmacol. 2:23-33, 2002). Treatments targeting this system, based on strong preclinical evidence, offer a completely new and promising approach for treating stress-related illnesses.

### BRIEF SUMMARY OF THE INVENTION

In one embodiment, the present invention is a method of treating a patient comprising the step of reducing the amount of mRNA encoding corticotropin-releasing factor receptor, wherein a mRNA encoding corticotropin-releasing factor receptor is destroyed and wherein the amount of corticotrophin releasing factor receptor is reduced. Preferably, the reduction of mRNA is by the use of a pharmaceutically effective dose of a nucleic acid enzyme.

Most preferably, the nucleic acid enzyme is a DNA enzyme.

In preferred versions of the present invention the nucleic acid enzyme is specific against corticotropin-releasing factor receptor 1 (CRF₁) or corticotropin-releasing factor receptor 2 (CRF₂).

In a preferred version of the present invention the treatment comprises the step of introducing a nucleic acid enzyme specific for corticotropin-releasing factor receptor into the patient's brain and a psychiatric illness such as anxiety or depression is treated.

In another embodiment, mRNA encoding CRF receptor is reduced in the periphery and the patient is being treated for an illness selected from the group consisting of irritable bowel syndrome (IBS), gastric esophageal reflux disease (GERD), gastric ulcers, and cardiovascular disease.

In another embodiment, the present invention is a composition comprising a pharmaceutically effective dose of a nucleic acid enzyme specific for CRF receptor.

Other objects, features, and advantages of the present invention will become apparent after evaluation of the specification, claims and drawings.

### BRIEF DESCRIPTION OF THE SEVERAL VIEWS OF THE DRAWINGS

FIG. 1 is a graph of ¹²⁵I-Sauvagine binding in rats treated with either saline or the CRF₁ nucleic acid enzyme.

FIGS. 2A and B are bar graphs describing percentage of open arm entries and percentage of open arm tune of rats treated with a control solution or CRF₁ nucleic acid enzyme. FIG. 2A describes injection into the basolateral amygdala (BLA) and FIG. 2B describes injection into the central nucleus of amygdala (CeA).

FIG. 3 is a bar graph describing the relative CRF_{2α} protein levels in control rats and rats treated with the CRF₂ nucleic acid enzyme.

FIG. 4 is a bar graph describing the percentage open arm time in rats treated with saline or 20 or 100 μg of a nucleic acid enzyme specific to CRF₂.

### DESCRIPTION OF THE INVENTION

In General

The present invention relates to the regulation of corticotropin-releasing factor (CRF) (Guillemin and Rosenberg, Endocrinology 57:599-607, 1955), which is a hormone and neurotransmitter thought to integrate the various electrophysiological, immune, endocrine and behavioral responses to stress (Arborelius, et al., J. Endocrinol. 160:1-12, 1999; Takahashi, Neurosci. Biobehav. Rev. 25:627-636, 2001). In general, the present invention is a method of reducing the amount of corticotropin-releasing factor receptor mRNA within a cell. Preferably, a nucleic acid enzyme targets the mRNA and destroys it, whereby the amount of corticotropin-releasing factor receptor that can be translated from mRNA is substantially reduced.

Studies in animals demonstrate that antagonism of the CRF system blocks the distress and physical effects related to stress (Takahashi, et al., supra, 2001; Bakshi, et al., J. Neurosci. 22:2926-2935, 2002). Studies in humans show that the CRF system in the brain is overactive in patients with depression, anxiety disorders and other neuropsychiatric problems (Nemeroff, J. Clin. Psychiatry 50:13-20, 1989; Chappell, et al., Biol. Psychiatry 39:776-783, 1996; Fossey, et al., Biol. Psychiatry 39:703-707, 1996; Bremner, et al., Am. J. Psychiatry 154:624-629, 1997; Mitchell, supra, 1998; Baker, e al., Am. J. Psychiatry 156:585-588, 1999). In addition, human and animal studies demonstrate that many effective antidepressant treatments decrease brain CRF activity (Veith, et al., Psychiatry Res. 46: 1-8, 1993). Based on these findings the pharmaceutical industry is currently intensively searching for compounds that will block or reduce the effects of CRF in the brain. Already some compounds have been identified and are in the early stages of human studies (Zobel, et al., J. Psychiatr. Res. 34:171-181, 2000).

The CRF system is now known to consist of at least seven components. CRF is a neurotransmitter that is released from neurons and has its effects by interacting with CRF receptors located on adjacent brain cells. Urocortin, urocortin II and urocortin III are other neurotransmitters similar to CRF that also interact with the system (Vaughan, et al., Nature 378:287-292, 1995; Lewis, et al., Proc. Natl. Acad. Sci. USA 98:7570-7575, 2001; Reyes, et al., Proc. Natl. Acad. Sci. USA 98:2843-2848, 2001). Once stimulated the receptors activate intracellular processes which mediate the stress effects.

CRF produces its effects by interacting with two different receptors termed CRF₁ and CRF₂ (Chen, et al., Proc Natl. Acad. Sci. USA 90:8967-8971, 1993; Perrin, et al., Proc. Natl. Acad. Sci. USA 92:2969-2973, 1995). There also exist at least three different splice variants of the CRF₂ receptor, termed “CRF_{2α},” “CRF_{2β}” and “CRF_{2γ};” (Lovenberg, et al., Proc. Nat. Acad. Sci. USA 92:836-840, 1995; Kostich, et al., Mol. Endocrinol. 12:1077-1085, 1998). In addition to CRF₁ and CRF₂ receptors, there also exists a protein, termed “CRF binding protein”, that is found in brain cells and functions to inactivate CRF after CRF is released (Potter, et al., Nature 349:423-425, 1991). In the Examples below, “R1” and “R2” are used to denote “CRF₁” and “CRF₂.” For a known variants of CRF₂, the current accepted terminology is CRF₂₍ₐ₎, CRF_{2(b)}, and CRF_{2(c)}. This application contains both the older and the newer terminology. In addition, many people refer to CRF as CRH (corticotrophin releasing hormone).

While much is known about the biology of CRF, considerably less is understood about CRF₁ and CRF₂. Most believe that the CRF₁ is responsible for mediating the effects of stress and also may be important in depression and anxiety (Makino, et al., Endocrinology 136:4517-4525, 1995; Aguilera, et al., J. Endocrinol. 153:185-191, 1997; Bonaz and Rivest, Am. J. Physiol. 275:R1438-R1449, 1998). However, other evidence suggests that CRF₂ may also play a critical role in mediating the effects of stress (Makino, et al., Neuroendocrinology 70:160-167, 1999; Bakshi, et al., supra, 2002). The pharmaceutical industry has targeted CRF₁ for the development of antagonists to block the effects of stress (Zobel, et al., supra, 2000). While interest in CRF₂ may exist, small molecule antagonists specific for this receptor remain to be discovered.

The present invention invokes a different therapeutic approach aimed at altering the levels of mRNA encoding CRF receptors and has the potential to be an effective strategy in the treatment of anxiety, depression and other stress-related problems. This approach is based on the hypothesis that the primary problem in these illnesses is over-expression of CRF and/or its receptors. Thus, a treatment aimed at the primary cause of these problems should prove more effective and be without non-specific effects on other systems. For example, drugs that control the regulation of CRF or its receptors would allow greater precision in stress management. Traditional approaches suffer from numerous unwanted effects because receptor antagonists affect all receptors throughout the brain and body, and do not selectively interact with those brain regions or organs that are most important in an illness.

### Nucleic Acid Enzyme

A particularly preferred form of the present invention uses nucleic acid enzymes to block the translation or to eliminate CRF receptor mRNA. The capacity of RNAs to catalyze chemical reactions in a conformation-dependent manner has been known for two decades (Kruger, et al., Cell 31:147-157, 1982). In recent years, catalytic nucleic acids composed entirely of DNA have been generated by in vitro selection strategies. These molecules ideally combine the catalytic activity of ribozymes with the stability of oligodeoxynucleotides. Most of the first nucleic acid enzymes proved inefficient at cleaving target sequences consisting of a more biologically relevant all-RNA substrate.

Recently, a highly efficient RNA-cleaving phosphodiester-linked nucleic acid enzyme was identified from a population of 1014 different DNA molecules after multiple successive rounds of selective amplification in vitro (Santoro and Joyce, Proc. Natl. Acad. Sci. USA 94:4262-4266, 1997). The “10-23” nucleic acid enzyme, so called because it was the 23^{rd} clone of the 10^{th} cycle of in vitro selection; comprises a divalent metal cation dependent catalytic domain comprised of 15 deoxynucleotides, flanked by two substrate recognition arms each composed of 8 bases that bind to the target RNA through Watson-Crick base-pairing. The nucleic acid enzyme cleaves a specific phosphodiester linkage between an unpaired purine (A, G) and a paired pyrimidine (C, U). Nucleic acid enzymes based on the 10-23 design may be custom made to target specific sites in virtually any mRNA, with sequence specificity conferred by the bases in the recognition arms. Such nucleic acid enzymes can discriminate between even subtle differences in RNA sequences (Cairns, et al., Nucleic Acids Res. 28:E9, 2000).

By “nucleic acid enzyme” it is meant a nucleic acid molecule capable of catalyzing reactions including, but not limited to, site-specific cleavage and/or ligation of other nucleic acid molecules, cleavage of peptide and amide bonds, and trans-splicing. Such a molecule with endonuclease activity may have complementarity in a substrate binding region to a specified gene target, and also has an enzymatic activity that specifically cleaves RNA or DNA in that target. That is, the nucleic acid molecule with endonuclease activity is able to intramolecularly or intermolecularly cleave RNA or DNA and thereby inactivate a target RNA or DNA molecule. This complementarity functions to allow sufficient hybridization of the enzymatic oligonucleotide molecule to the target RNA or DNA to allow the cleavage to occur. 100% complementarity is preferred, but complementarity as low as 50-75% may also be useful in this invention. The nucleic acids may be modified at the base, sugar, and/or phosphate groups.

The term nucleic acid enzyme is used interchangeably herein with phrases such as ribozymes, catalytic RNA, enzymatic RNA, catalytic DNA, catalytic oligonucleotides, nucleozyme, DNAzyme, RNA enzyme, endoribonuclease, endonuclease, minizyme, leadzyine, oligozyme or nucleic acid enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving activity to the molecule.

By “enzymatic portion” or “catalytic domain” is meant that portion/region of the enzymatic nucleic acid essential for cleavage of a nucleic acid substrate.

By “substrate binding arm” or “substrate binding domain” is meant that portion/region of a nucleic acid enzyme which is complementary to (i.e., able to base-pair with) a portion of its substrate. Generally, such complementarity is 100%, but can be less if desired. For example, as few as; 12 bases out of 16 may be base-paired. That is, these arms contain sequences within a nucleic acid enzyme which are intended to bring the enzyme and target RNA together through complementary base-pairing interactions. The nucleic acid enzyme of the invention may have binding arms that are contiguous or non-contiguous and may be of varying lengths. The length of the binding arm(s) is preferably greater than or equal to four nucleotides; specifically 12-100 nucleotides; more specifically 14-24 nucleotides long. If two binding arms are chosen, the design is such that the lengths of the binding arms are symmetrical (i.e., each of the binding arms is of the same length; e.g., five and five nucleotides, six and six nucleotides or seven and seven nucleotides long) or asymmetrical (ie., the binding arms are of different length; e.g., six and three nucleotides; three and six nucleotides long; four and five nucleotides long; four and six nucleotides long; four and seven nucleotides long; and the like). By “complementarity” is meant a nucleic acid that can form hydrogen bond(s) with another RNA sequence by either traditional Watson-Crick or other non-traditional types (for example, Hoogsteen type) of base-pair interactions.

In a preferred embodiment the invention discloses a class of enzymatic cleaving agents which exhibit a high degree of specificity for the RNA of CRF receptors. The enzymatic nucleic acid molecule is preferably targeted to a highly conserved sequence region of target mRNAs encoding CRF receptor proteins such that specific treatment of a disease or condition can be provided with either one or several enzymatic nucleic acids. Such enzymatic nucleic acid molecules can be delivered exogenously to specific cells as required. Alternatively, the nucleic acid enzymes can be expressed from DNA/RNA vectors that are delivered to specific cells. Such nucleic acid enzymes are useful for the prevention of the diseases and conditions discussed above, and any other diseases or conditions that are related to the levels of CRF receptor activity in a cell or tissue.

By “related” is meant that the inhibition of CRF receptor RNAs and thus reduction in the level respective receptor activity will relieve to some extent the symptoms of the disease or condition.

Thus, in a first aspect, the invention features nucleic acid enzymes that inhibit gene expression and/or receptor function. These chemically or enzymatically synthesized RNA/DNA molecules contain substrate binding domains that bind to accessible regions of their target mRNAs. The RNA/DNA molecules also contain domains that catalyze the cleavage of RNA. The DNA molecules are preferably nucleic acid enzymes of the 10-23 motif. The RNA molecules are preferably ribozymes of the hammerhead or hairpin motif. Upon binding, the enzymatic oligonucleotides cleave the target mRNAs, preventing translation and protein accumulation. In the absence of the expression of the target gene, CRF receptor gene expression and/or function is inhibited.

By “patient” is meant an organism which is a donor or recipient of explanted cells or the cells themselves. “Patient” also refers to an organism to which enzymatic nucleic acid molecules can be administered. Preferably, a patient is a mammal or mammalian cells. More preferably, a patient is a human or human cells.

By “vectors” is meant any nucleic acid- and/or viral-based technique used to deliver a desired nucleic acid.

These nucleic acid enzymes, individually, or in combination or in conjunction with other drugs, can be used to treat diseases or conditions discussed above. For example, to treat a disease or condition associated with CRF receptor levels, the patient may be treated, or other appropriate cells may be treated, as is evident to those skilled in the art.

Synthesis of nucleic acids greater than 100 nucleotides in length is difficult using automated methods, and the therapeutic cost of such molecules is prohibitive. In this invention, small nucleic acid motifs (e.g., 10-23 nucleic acid enzymes, hammerhead or the hairpin ribozymes) are used for exogenous delivery. The simple structure of these molecules increases the ability of the nucleic acid to invade targeted regions of the mRNA structure. However, these nucleic acid molecules can also be expressed within cells from eukaryotic promoters (e.g., Izant and Weintraub, Science 229:345, 1985; McGarry and Lindquist, Proc. Natl. Acad. Sci. USA 83:399, 1986; Sullenger-Scanlon, et al., Proc. Natl. Acad. Sci. USA 88:10591-10595, 1991; Kashani-Sabet, et al., Antisense Res. Dev. 2:3-15, 1992; Dropulic, et al., J. Virol. 66:1432-1441, 1992; Weerasinghe, et al., J. Virol. 65:5531-5534, 1991; Ojwang, et al., Proc. Natl. Acad. Sci. USA 89:10802-10806, 1992; Chen, et al., Nucleic Acids Res. 20:4581-4589, 1992; Sarver, et al., Science 247:1222-1225, 1990; Thompson, et al., Nucleic Acids Res. 23:2259, 1995). Those skilled in the art realize that any nucleic acid can be expressed in eukaryotic cells from the appropriate DNA/RNA vector. The activity of such nucleic acids can be augmented by their release from the primary transcript by a ribozyme (Draper, et al., PCT W093/23569, and Sullivan, et al., PCT W094/02595; Ohkawa, et al., Nucleic Acids Symp. Ser. 27:15-6, 1992; Taira, et al., Nucleic Acids Res. 19:5125-5130, 1991; Ventura, et al., Nucleic Acids Res. 21:3249-3255, 1993; Chowrira, et al., J. Biol. Chem. 269:25856, 1994).

Therapeutic nucleic acid enzymes delivered exogenously must optimally be stable within cells until translation of the target RNA has been inhibited long enough to reduce the levels of the undesirable protein. This period of time varies between hours to days depending upon the disease state. Clearly, enzymatic nucleic acids must be resistant to nucleases in order to function as effective intracellular therapeutic agents. Improvements in the chemical synthesis of RNA and DNA (Wincott, et al., Nucleic Acids Res. 23:2677, 1995) have expanded the ability to modify enzymatic nucleic acids by introducing nucleotide modifications to enhance their nuclease stability as described above.

“Nucleotide” as used herein is as recognized in the art to include natural bases (standard), and modified bases well known in the art. Such bases are generally located at the 1′ position of a sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein, et al., International PCT Publication No. WO 92/07065; Usman, et al., International PCT Publication No. WO 93/15187; all hereby incorporated by reference herein). There are several examples of modified nucleic acid bases known in the art, which have been recently summarized (Limbach, et al., Nucleic Acids Res. 22:2183, 1994). Some of the non-limiting examples of base modifications that can be introduced into enzymatic nucleic acids without significantly affecting their catalytic activity include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine) and others (Burgin, et al., Biochemistry 35:14090, 1996). By “modified bases” in this aspect is meant nucleotide bases other than adenine, guanine, cytosine and uracil at 1′ position or their equivalents; such bases may be used within the catalytic core of the enzyme and/or in the substrate-binding regions.

Target mRNA Sequences

To design a nucleic acid enzyme one must know the target mRNA sequence. Most mRNA sequences for the CRF system are available publicly through the GenBank database. For example, the GenBank accession number for human CRF₁ is NM_{—}004382, for human CRF_{2α} is U34587, for CRF_{2β} is AF011406 and for CRF_{2γ}, is AF019381. One skilled in the art would realize that there are often many accession numbers for a given mRNA sequence, and additional accession numbers are being generated on a continuing basis. The various accession numbers are often deposited from different investigators. The different deposited sequences are usually identical, but variations do occur due to experimental error, natural variability in the population, and most often due to the level of completeness of the sequencing project. One skilled in the art would realize that using any experimentally validated sequence would be acceptable for choosing the target recognition sequence when designing enzymatic nucleic acids.

To create the substrate binding, one must know 17 base pairs, preferably near the translation start site such that the 9^{th} base pair is a purine (AG) and the 10^{th} is a pyrimidine (UC). In the Examples below, the first 8 bases of the nucleic acid enzyme are antisense to bases 10-17 of the target mRNA. The next 15 bases of the nucleic acid enzyme correspond to the sequence of the common catalytic domain described previously (Santoro and Joyce, 1997). The final 8 bases of the nucleic acid enzyme are antisense to bases 1-8 of the target mRNA. This leaves base 9 of the mRNA unpaired allowing efficient cleavage of the target mRNA.

In a preferred version of the present invention we have disclosed a 10-23 nucleic acid enzyme targeted specifically against the corticotropin releasing factor receptor mRNA (Santoro and Joyce, supra, 1998). The nucleic acid enzyme will selectively bind to a CRF receptor mRNA and cleave it near the translation start site. The cleaved CRF receptor mRNA will not be translated leading to a reduction in the CRF receptor protein levels.

We have specifically targeted the translation start sites of the CRF₁ and CRF₂ mRNAs with the following DNA enzymes: CRF₁ DNA enzyme sequence (5-3) is GCCCTCCCAGGCTAGCTACAACGACCTCGGGCT. CRF₂ DNA enzyme sequence (5-3) is CGCGTCCAGGCTAGCTACAACGATGCGCTCC. The first and last 8 bases are binding sequences. One skilled in the art would realize that enzymatic nucleic acids can be targeted against any region of a particular mRNA, and that targeting other regions of an mRNA may also beneficially reduce mRNA levels. Separately, on skilled in the art would realize that using multiple enzymatic nucleic acids in combination with each other may increase efficacy of the treatment. Furthermore, the present invention includes using different classes of enzymatic nucleic acids in combination with each other (for example ribozymes with DNA enzymes), and different modes of delivery in combination with each other (for example direct administration of a DNA enzyme in combination with a vector expressing a ribozyme).

As mentioned earlier, subtypes have been described for the CRF receptors. For example, there are at least three subtypes of the CRF₂ receptor. These receptor subtypes differ in where they are expressed in the body and in what physiological processes they regulate. Currently available antagonists for the CRF receptors do not differentiate significantly between the different subtypes. Because the DNA sequence differs between the different receptor subtypes, one potential advantage of the nucleic acid enzyme is that is can be designed to target the mRNA corresponding to a specific subtype of CRF₁ or CRF₂ receptor. For example, a nucleic acid enzyme could be designed to target the region of the CRF_{2α}RNA that differs from the CRF_{2β} and CRF_{2γ}RNAs. This would result in the selective reduction of the mRNA and corresponding protein for only the CRF_{2α} without affecting the other subtypes of the CRF₂ receptor. This subtype specificity available with a nucleic acid enzyme would decrease the potential for unwanted toxicities that result from reduction of the other subtypes.

### Administration

Methods of administration can include oral, subcutaneous, sublingual, intravenous, intranasal, intrathecal, intra-arterial, transdermal and suppository. Preferred specific targets include the patient's brain, most particularly the amygdala (BLA or CeA) section and lateral septum. There are many other areas known to be involved in the psychopathology of stress related disorders including, but not limited to, orbito frontal cortex, thalamus, paraventricular nucleus of the thalamus, lateral septum, hypothalamus, brainstem, etc. CRF receptors are located in many other regions of the CNS that have not yet been directly implicated in psychopathology, but may well be involved.

In another preferred embodiment, the nucleic acid enzyme is delivered to the patient's periphery. By “periphery” we mean any tissue that is not part of the CNS.

Because these agents can be used to treat several diseases which involve several tissue types, the absolute amount of reduction of mRNA and corresponding protein that is adequate will depend on tissue type and the disease. In general, reduction will be deemed adequate if it imparts a beneficial outcome.

Sullivan, et al., PCT WO 94/02595, describes the general methods for delivery of enzymatic nucleic acid molecules. Nucleic acid enzymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, nucleic acid enzymes may be directly delivered ex vivo to cells or tissues with or without the aforementioned vehicles. Alternatively, the DNA/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of enzymatic-nucleic acid delivery and administration are provided in Sullivan, et al., supra and Draper, et al., PCT W093/23569 which have been incorporated by reference herein.

In a preferred embodiment, nucleic acid enzymes are added directly, or can be complexed with cationic lipids, packaged within liposomes, or otherwise delivered to target cells. The nucleic acid or nucleic acid complexes can be locally administered to relevant tissues ex vivo, or in vivo through injection, infusion pump or stent, with or without their incorporation in biopolymers. In another preferred embodiment, the nucleic acid enzyme is administered to the site of CRF receptor activity (e.g., lateral septum) in an appropriate liposomal vehicle.

In another aspect of the invention, nucleic acid enzymes that cleave target molecules and inhibit CRF receptor activity are expressed from transcription units inserted into DNA or RNA vectors. The recombinant vectors are preferably DNA plasmids or viral vectors. Nucleic acid enzyme-expressing viral vectors could be constructed based on, but not limited to, adeno-associated virus, retrovirus, adenovirus, or alphavirus. Preferably, the recombinant vectors capable of expressing the nucleic acid enzymes are delivered as described above, and persist in target cells. Alternatively, viral vectors may be used that provide for transient expression of nucleic acid enzymes. Such vectors might be repeatedly administered as necessary. Once expressed, the nucleic acid enzymes cleave the target mRNA. Delivery of nucleic acid enzyme expressing vectors could be systemic, such as by intravenous or intramuscular administration, by administration to target cells ex-planted from the patient followed by reintroduction into the patient, or by any other means that would allow for introduction into the desired target cell (for a review see Couture and Stinchcomb, 1996, TIG., 12, 510). In another aspect of the invention, nucleic acid enzymes that cleave target molecules and inhibit CRF receptor function are expressed from transcription units inserted into DNA, RNA, or viral vectors. Preferably, the recombinant vectors capable of expressing the nucleic acid enzymes are locally delivered as described above, and transiently persist in neuronal cells. However, other mammalian cell vectors that direct the expression of DNA may be used for this purpose.

The molecules of the instant invention can be used as pharmaceutical agents. Pharmaceutical agents prevent, inhibit the occurrence, or treat (alleviate a symptom to some extent, preferably all of the symptoms) a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a lipid or liposome delivery mechanism, standard protocols for formulation can be followed. The compositions of the present invention may also be formulated and used as tablets, capsules or elixirs for oral administration; suppositories for rectal administration; sterile solutions; suspensions for injectable administration; and the like.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the central nervous system. A liposome formulation which can facilitate the association of drug with active transport molecules on the surface of the blood brain barrier, such as, the manose and galactose transporter is also useful. This approach may provide enhanced delivery of the drug to central nervous system cells by taking advantage of the efficiency of the transporters to deliver sugars to the brain.

The invention also features the use of a composition comprising surface-modified liposomes containing poly(ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic, et al., Chem. Rev. 95:2601-2627, 1995; Ishiwata, et al., Chem. Pharm. Bull. 43:1005-1011, 1995). Such liposomes have been shown to accumulate selectively in tumors presumably by extravasation and capture in the neovascularized target tissues (Lasic, et al., Science 267:1275-1276, 1995; Oku, et al., Biochim. Biophys. Acta 1238:86-90, 1995). The long-circulating liposomes enhance the pharmacokinetics and pharmacodynamics of DNA and RNA, particularly compared to conventional cationic liposomes which are known to accumulate in tissues of the MPS (Liu, et al., J. Biol. Chem. 42:24864-24870, 1995; Choi, et al., International PCT Publication No. WO 96/1.0391; Ansell, et al., International PCT Publication No. WO 96/10390; Holland, et al., International PCT Publication No. WO 96/10392). All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives; stabilizers, dyes and flavoring agents may be provided. Id. at 1449. These include sodium benzoate, sorbic acid and esters of p-hydroxybenzoic acid. In addition, antioxidants and suspending agents may be used.

A pharmaceutically effective dose is that dose required to prevent, inhibit the occurrence, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration; concurrent medication, and other factors which those skilled in the medical arts will recognize. Generally, an amount between 0.1 mg/kg and 100 mg body weight/day of enzymatic nucleic acid is administered dependent upon potency of the negatively charged polymer.

Alternatively, the enzymatic nucleic acid molecules of the instant invention can be expressed within cells from eukaryotic promoters (e.g., Izant and Weintraub, supra, 1985; McGarry and Lindquist, supra, 1986; Scanlon, et al., Proc. Natl. Acad. Sci. USA 88:10591-1-10595, 1991; Kashani-Sabet, et al., supra, 1992; Dropulic, et al., supra, 1992; Weerasinghe, et al., supra, 1991; Ojwang, et al., supra, 1992; Chen, et al., supra, 1992; Sarver, et al., supra, 1990; Thompson, et al., supra, 1995; Good, et al., Gene Therapy 4:45, 1997). Those skilled in the art realize that any nucleic acid can be expressed in eukaryotic cells from the appropriate DNA/RNA vector. The activity of such nucleic acids can be augmented by their release from the primary transcript by a ribozyme (Draper, et al., PCT WO 93/23569, and Sullivan, et al., PCT WO 94/02595; Ohkawa, et al., supra, 1992; Taira, et al., supra, 1991; Ventura, et al., supra, 1993; Chowrira, et al., supra, 1994).

In another aspect of the invention, enzymatic nucleic acid molecules that cleave target molecules are expressed from transcription units (see for example Couture, et al., TIG., 12:510, 1996) inserted into DNA or RNA vectors. The recombinant vectors are preferably DNA plasmids or viral vectors. Nucleic acid enzyme expressing viral vectors could be constructed-based on, but not limited to, adeno-associated virus, retrovirus, adenovirus, or alphavirus. Preferably, the recombinant vectors capable of expressing the nucleic acid enzymes are delivered as described above, and persist in target cells. Alternatively, viral vectors may be used that provide for transient expression of Nucleic acid enzymes. Such vectors might be repeatedly administered as necessary. Once expressed, the nucleic acid enzymes cleave the target mRNA. The active nucleic acid enzyme contains an enzymatic center or core equivalent to those in the examples, and binding arms able to bind target nucleic acid molecules such that cleavage at the target site occurs. Other sequences may be present which do not interfere with such cleavage. Delivery of nucleic acid enzyme expressing vectors could be systemic, such as by intravenous or intramuscular administration, by administration, to target cells ex-planted from the patient followed by reintroduction into the patient, or by any other means that would allow for introduction into the desired target cell (for a review see Couture, et al., supra, 12:510, 1996).

In another aspect of the invention, the expression vector comprises: a transcription initiation, region (e.g., eukaryotic pol I, II or III initiation region); b) a transcription termination region (e.g., eukaryotic pol I, II or III termination region); c) a gene encoding at least one of the nucleic acid catalyst of the instant invention; and wherein said gene is operably linked to said initiation region and said termination region, in a manner which allows expression and/or delivery of said nucleic acid, molecule. The vector may optionally include an open reading frame (ORF) for a protein operably linked on the 5′ side or the 3′-side of the gene encoding the nucleic acid catalyst of the invention; and/or an intron (intervening sequences).

Transcription of the enzymatic nucleic acid sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, Proc. Natl. Acad. Sci. USA 87:6743-6747, 1990; Gao and Huang, Nucleic Acids Res. 21:2867-2872, 1993; Lieber, et al., Methods Enzymol. 217:47-66, 1993; Zhou, et al., Mol. Cell. Biol. 10:4529-4537, 1990). Several investigators have demonstrated that ribozymes expressed from such promoters can function in mammalian cells (e.g. Kashani-Sabet, et al., supra, 1992; Ojwang, et al., supra, 1992; Chen, et al., supra, 1992; Yu, et al., Proc. Natl. Acad. Sci. USA 90:6340-6344, 1993; L'Huillier, et al., EMBO J. 11:4411-4418, 1992; Lisziewicz, et al., Proc. Natl. Acad. Sci. USA 90:8000-8004, 1993; Thompson, et al., Nucleic Acids Res. 23:2259, 1995; Sullenger and Cech, Science 262:1566, 1993). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson, et al., supra; Couture and Stinchcomb, supra, 1996; Noonberg, et al., Nucleic Acid Res. 22:2830, 1994; Noonberg, et al., U.S. Pat. No. 5,624,803; Good, et al., supra, 1997; Beigelman, et al., International PCT Publication No. WO 96/18736). The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, supra, 1996). Similarly, a nucleic acid enzyme transcription unit can be incorporated into a variety of single stranded DNA expression vectors (Tan, et al., Biochemistry 43:1111, 2004; Chen, et al., Biotechniques 34:167, 2003; Chen and McMicken, Gene Ther. 10:1776, 2003). These vectors are able to generate sufficient copies of nucleic acid enzyme molecules to significantly reduce target mRNA levels.

In yet another aspect the invention features an expression vector comprising a nucleic acid sequence encoding at least one of the nucleic acid enzymes of the invention in a manner which allows expression of that nucleic acid molecule. The expression vector comprises in one embodiment; a) a transcription initiation region; b) a transcription termination region; c) a gene encoding at least one said nucleic acid molecule; and wherein said gene is operably linked to said initiation region and said termination region, in a manner which allows expression and/or delivery of said nucleic acid molecule. In another preferred embodiment the expression vector comprises: a) a transcription initiation region; b) a transcription termination region; c) an open reading frame; d) a gene encoding at least one said nucleic acid molecule, wherein said gene is operably linked to the 3′-end of said open reading frame; and wherein said gene is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a gene encoding at least one said nucleic acid molecule; and wherein said gene is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a gene encoding at least one said nucleic acid molecule, wherein said gene is operably linked to the 3′-end of said open reading frame; and wherein said gene is operably linked to said initiation region, said intron, said open reading frame and said termination region, in a manner which allows expression and/or delivery of said nucleic acid molecule.

### Uses of Nucleic Acid Enzymes

Another potential use for the nucleic acid enzymes would be as a tool to study the effects of reducing CRF receptor levels. More importantly, given that current research indicates that over-stimulation of the CRF receptors may, in part, be responsible for anxiety disorder and/or depression, the CRF receptor nucleic acid enzymes are likely to be an effective treatment for these conditions and other psychiatric and neurological conditions. Disorders to be treated include those of the brain such as depression, anxiety and all other neurological and psychiatric illnesses. In addition, illnesses in the periphery, the pathophysiology of which may be related to the CRF system can be targeted. These include such illnesses as irritable bowel syndrome (IBS), gastric esophageal reflux disease (GERD), gastrointestinal ulcers, cardiovascular disease and numerous others.

### EXAMPLES

### Example 1

### Infusion of a Nucleic Acid Enzyme Specific for CRF₁ into Rat Brain

### Enzyme Infusion

Male Sprague-Dawley rats were equipped with bilateral stainless steel guide cannulae aimed at either the central-nucleus (CeA) or the basolateral nucleus (BLA) of the amygdala. Stereotaxic coordinates (based on the atlas of Paxinos and Watson, 1986) were as follows: CeA—anterior/posterior plane =−2.0 mm from bregma; lateral/medial plane =±4.0 mm from midline; dorsal/ventral plane =−5.2 mm from skull surface. BLA—anterior/posterior plane =−3.0 mm from bregma; lateral/medial plane =±5.0 mm from midline; dorsal/ventral plane =−5.8 mm from skull surface. Two weeks after surgeries, injectors were lowered through the guide cannulae to the final dorsal/ventral coordinates (−8.2 mm for the CeA, and −8.8 for the BLA), and each side received 20 μg of the CRF₁ receptor nucleic acid enzyme infused at a concentration of 20 μg/μl and at a rate of 0.65 μl/min. This procedure was carried out once a day for 5 consecutive days; control animals for each brain region received the same regimen of infusions, but received isotonic saline instead of the nucleic acid enzyme.

### Behavioral Testing

Two hours after the 5^{th} infusion, all rats were tested for anxiety-like responses in the elevated-plus maze. This apparatus consists of 2 open arms (no walls) in which the lighting level is bright, and 2 closed arms (with high walls enclosing the arm) in which the lighting level is relatively dark. Normally, rats prefer to spend the majority of their time in the closed, presumably “safer feeling”, arms. A relative increase in the time spent in the open arms is thought to indicate an increase in exploratory approach behaviors, and is generally interpreted to indicate a decrease in the fearful state of the animal. Each rat was tested individually in this paradigm by being placed in the center of the maze, and then being allowed 5 minutes to explore the maze.

### Receptor Binding Assay

Approximately one hour following behavioral testing rats were sacrificed by decapitation and the amygdala was removed using a tissue punch from 2 mm thick sections and stored at −80° C. until assay. Tissue from two rats was pooled and CRF receptor levels were quantified using [¹²⁵I]tyr⁰sauvagine (2200 Ci/mmole) following a published protocol (Roseboom, et al., Brain Res. Mol. Brain Res. 92:115-127, 2001).

### Characterizing the Effects of a Nucleic Acid Enzyme Targeting CRF₁ Receptors (CRF₁ In Vitro Expression

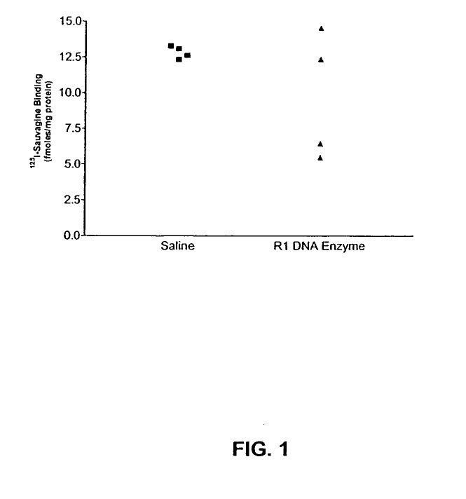

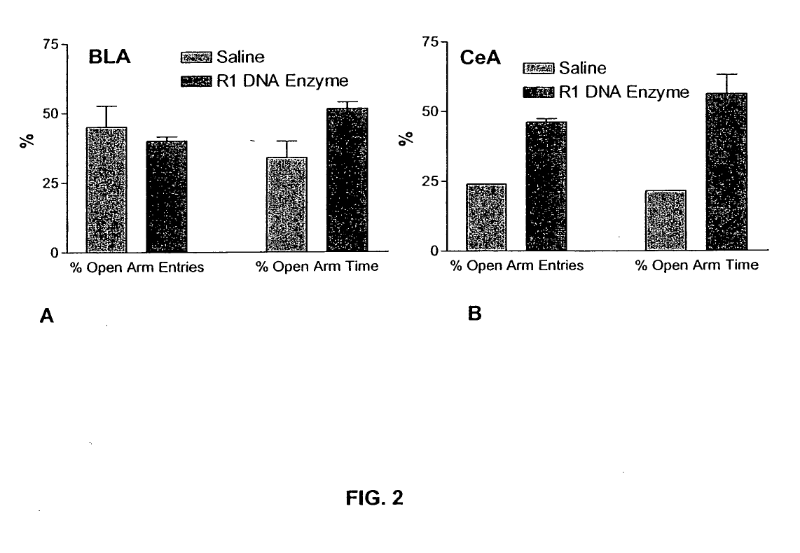

Referring to FIGS. 1 and 2, analysis of CRF receptor levels revealed that treatment with the nucleic acid enzyme dramatically reduced receptor levels in two of the four pairs of animals. This indicates that the CRF₁ nucleic acid enzyme is effective in reducing the expression of CRF receptors in the rat amygdala.

In a separate experiment, direct injection of the CRF₁ nucleic acid enzyme into two subnuclei of the rat amygdala also affects rat behavior. Injection of the CRF₁ nucleic acid enzyme into the basolateral amygdala (BLA), a region known to express R1 receptors, appears to increase the percent time treated rats spend in the open arms of the elevated plus maze. Furthermore, injection of the CRF₁ nucleic acid enzyme into the central nucleus of the amygdala, another region known to express R1 receptors, increases both the percent time and the percent entries into the open arm of the elevated plus maze. The use of the elevated plus maze has been a well characterized behavioral test, and increased time or entries into the open arm is indicative of a less anxious or less fearful rat. Thus, it appears the CRF₁ nucleic acid enzyme is capable of reducing receptor levels and this reduction correlates with a change in behavior. In conclusion, the data suggest that the CRF₁ nucleic acid enzyme may prove to be an important therapeutic agent in the treatment of anxiety disorder.

### Example 2

### Characterizing the Effects of a Nucleic Acid Enzyme Targeting CRF_{2α} Receptors on CRF_{2α}In Vitro Expression

In order to test the effectiveness of the CRF_{2□} nucleic acid enzyme, we made use of the TnT Coupled Reticulate Lysate in vitro transcription/translation system (Promega, Madison, Wis.). In this system a linearized full-length human CRF_{2□} cDNA was used as template. The TnT system, in a single reaction tube, will both transcribe mRNA from the template and translate the mRNA into protein. Test reactions included 1 μg of a nucleic acid enzyme designed against the human CRF_{2□}mRNA. All reactions included ³⁵S methionine so that the amount of translated protein could be determined. Completed reactions were run on SDS-PAGE gels, the gels were dried on to Whatman paper, exposed to phosphorimaging screens, and imaged using a STORM phosphorimager (Molecular Dynamics, NJ). The radioactive signal from the band corresponding to the CRF_{2□} protein was determined with the ImageQuant software (Molecular Dynamics). Control reactions (without nucleic acid enzyme) and test reactions (with nucleic acid enzyme) were performed 8 times each and run on 4 gels.

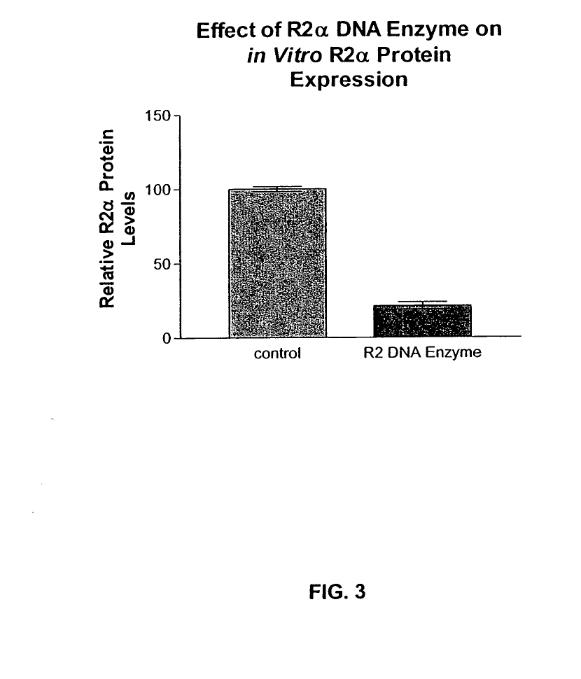

Referring to FIG. 3, our results demonstrate that the nucleic acid enzyme treated reactions greatly reduce (79% reduction) levels of the CRF_{2□} protein. This is a highly significant finding (p<0.001), and suggests that the nucleic acid enzyme is highly effective in preventing CRF_{2□} protein from being made.

### Example 3

### Infusion of a Nucleic Acid Enzyme Specific for CRF₂ into Rat Brain

### Enzyme Infusion

Male Sprague-Dawley rats were equipped with bilateral stainless steel guide cannulae aimed the lateral septum (LS). Stereotaxic coordinates (based on the atlas of Paxinos and Watson, 1986) were as follows: LS—anterior/posterior plane =+0.4 mm from bregma; lateral/medial plane =±0.8 mm from midline; dorsal/ventral plane =−5.0 mm from skull surface. One week after surgeries, injectors were lowered-through the guide cannulae to the final dorsal/ventral coordinates (−7.5 mm for the LS), and the R2 nucleic acid enzyme was infused at a concentration of 20 μg/μl/side or 100 μg/μl/side. This procedure was carried out once a day for 5 consecutive days; control animals for each brain region received the same regimen of infusions, but received isotonic saline instead of the nucleic acid enzyme.

### Behavioral Testing

Two hours after the 5^{th} infusion, all rats were tested for anxiety-like responses in the elevated-plus maze. This apparatus consists of 2 open arms (no walls) in which the lighting level is bright, and 2 closed arms (with high walls enclosing the arm) in which the lighting levels is relatively dark. Normally, rats prefer to spend the majority of their time in the closed, presumably “safer feeling”, arms. A relative increase in the time spent in the open arms is thought to indicate an increase in exploratory approach behaviors, and is generally interpreted to indicate a decrease in the fearful state of the animal. Each rat was tested individually in this paradigm by being placed in the center of the maze, and then being allowed 5 minutes to explore the maze.

### Characterization of R2 Nucleic Acid Enzyme Treatment on Behavior

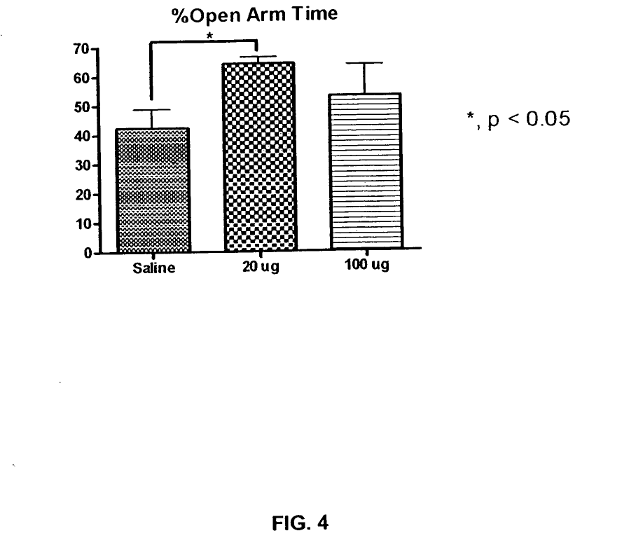

Referring to FIG. 4, direct injection of the R2 nucleic acid enzyme into the LS affects rat behavior. Injection of the R2 nucleic acid enzyme at 20 μg/μl/side into the LS, a region known to express R2 receptors, appears to significantly increase the percent time treated rats spend in the open arms of the elevated plus maze (see FIG. 4). The 100 μg/μl/side dose did not produce a significant change in percent open arm time. The use of the elevated plus maze has been a well characterized behavioral test, and increased time or entries into the open arm is indicative of a less anxious or less fearful rat. Thus, it appears the R1 nucleic acid enzyme is capable of reducing receptor levels and this reduction correlates with a change in behavior. In conclusion, the data suggest that the R2 nucleic acid enzyme is an important therapeutic agent in the treatment of anxiety disorders.

We claim:

1. A method of treating a patient comprising the step of reducing the amount of mRNA encoding corticotropin-releasing factor receptor, wherein a mRNA encoding corticotropin-releasing factor receptor is destroyed and wherein the amount of corticotrophin releasing factor receptor is reduced.

2. The method of claim 1 wherein the reduction of mRNA is by the use of a pharmaceutically effective dose of a nucleic acid enzyme.

3. The method of claim 2 wherein the nucleic acid enzyme is a DNA enzyme.

4. The method of claim 3 wherein the DNA enzyme is a 10-23 enzyme.

5. The method of claim 2 wherein the nucleic acid enzyme is specific against corticotropin-releasing factor receptor 1 (CRF₁).

6. The method of claim 5 wherein the nucleic acid enzyme is specific against a subtype of corticotropin-releasing factor receptor 1 (CRF₁).

7. The method of claim 2 wherein the nucleic acid enzyme is specific against corticotropin-releasing factor receptor 2 (CRF₂).

8. The method of claim 7 wherein the nucleic acid enzyme is specific against a subtype of corticotropin-releasing factor receptor 2 (CRF₂).

9. The method of claim 1 wherein the treatment comprises the step of introducing a nucleic acid enzyme specific for corticotropin-releasing factor receptor into the patient's brain.

10. The method of claim 9 wherein the nucleic acid enzyme is a DNA acid enzyme.

11. The method of claim 9, wherein a psychiatric illness such as anxiety or depression is treated.

12. The method of claim 1, wherein mRNA encoding CRF receptor is reduced in the periphery.

13. The method of claim 12 wherein the patient is being treated for an illness selected from the group consisting of irritable bowel syndrome (IBS), gastric esophageal reflux disease (GERD), gastric ulcers, and cardiovascular disease.
